(19) United States
(12) Patent Application Publication
(10) Pub. No.: US 2023/0389785 A1
(43) Pub. Date: Dec. 7, 2023

(54) ENDOSCOPE

(71) Applicant: FUJIFILM Corporation, Tokyo (JP)

(72) Inventors: Masaya INOUE, Kanagawa (JP); Takashi HARADA, Kanagawa (JP)

(73) Assignee: FUJIFILM Corporation, Tokyo (JP)

(21) Appl. No.: 18/454,810

(22) Filed: Aug. 24, 2023

Related U.S. Application Data
(63) Continuation of application No. PCT/JP2022/007580, filed on Feb. 24, 2022.

(30) Foreign Application Priority Data
Feb. 26, 2021 (JP) 2021-030647

(51) Int. Cl. A61B 1/018; A61B 1/00

(52) U.S. Cl. CPC A61B 1/018; A61B 1/00128; A61B 1/00103; A61B 1/00042

(57) ABSTRACT

A locking mechanism includes a locked member provided in an elevating operation lever and a spring member that applies a biasing force to the locked member. In a case where the elevating operation lever is not operated, the locked member is locked to a locking piece and the elevating operation lever is brought into a locked state, and in a case where the elevating operation lever is operated, a locked claw is separated from the locking piece by a lock release operation against the biasing force and the locked state of the elevating operation lever is released.

### CROSS-REFERENCE TO RELATED APPLICATIONS

This application is a Continuation of PCT International Application No. PCT/JP2022/007580 filed on 24 Feb. 2022, which claims priority under 35 U.S.C. § 119(a) to Japanese Patent Application No. 2021-030647 filed on 26 Feb. 2021. The above application is hereby expressly incorporated by reference, in its entirety, into the present application.

### BACKGROUND OF THE INVENTION

### 1. Field of the Invention

The present invention relates to an endoscope that changes a lead-out direction of a treatment tool.

### 2. Description of the Related Art

Endoscopes used in the medical field are used not only for observation of the inside of a subject but also for various treatments on an observation site. The endoscope comprises an insertion part to be inserted into the subject and an operation part installed consecutively to a proximal end portion of the insertion part. Various treatment tools, such as forceps and incision tools, are inserted into a treatment tool channel in the insertion part from a treatment tool lead-in port provided in the operation part of the endoscope and are led out of a treatment tool lead-out port opened at a distal end portion of the insertion part, so that various treatments, such as resection and collection of the observation site, are performed.

It is necessary to change a lead-out direction of the treatment tool led out from the treatment tool lead-out port of the endoscope in order to treat a desired position in the subject. For this purpose, an elevator that changes the lead-out direction of the treatment tool is provided at the distal end portion of the insertion part. The posture of the elevator is moved between a fallen position and an elevated position by operating an elevating operation lever provided on the operation part. The elevator can guide the treatment tool and change the lead-out direction of the treatment tool by moving the elevator from the fallen position to the elevated position (see JP2020-137947A).

Additionally, in an endoscope described in JP2006-015018A (corresponding to US2007/0232857A1), a slit into which the guide wire is fitted is formed in an elevator. The guide wire is inserted into a treatment tool channel together with a treatment tool and guides the treatment tool. Then, in a case where the elevator is at an elevated position, the guide wire is fitted into the slit of the elevator and the elevator is fixed. Accordingly, the elevating operation lever that is interlocked with the elevator is also fixed.

Meanwhile, in an endoscope described in JP2011-072455A (corresponding to US2011/0077461A1), a friction resistance applying spring material is attached to an elevating operation lever. The friction resistance applying spring material rotates integrally with the elevating operation lever in a state where the friction resistance applying spring material is pressed against a fixed wall formed on an operation part. A lubricant is applied between the friction resistance applying spring material and the fixed wall. The friction resistance applying spring material is pressed against the fixed wall by an elastic force, and friction resistance is generated. In a case where a user releases the hand from the elevating operation lever, the elevating operation lever is temporarily stopped by the friction resistance.

### SUMMARY OF THE INVENTION

In a case where a treatment is performed using an endoscope, a user operates an elevating operation lever with one hand to change a lead-out direction of a treatment tool and reliably stops an elevator in the middle of an elevated position and a fallen position to perform various treatments.

However, in the endoscopes described in JP2020-137947A and JP2006-015018A, it is not considered that the elevating operation lever is operated with one hand and the elevator is stopped in the middle of the elevated position and the fallen position. In particular, in the endoscope described in JP2006-015018A, only the guide wire is fixed such that the elevator and the elevating operation lever are stopped in a case where the elevator is at the elevated position. That is, since the elevating operation lever cannot be fixed at a position other than the elevated position, it is difficult to operate the elevating operation lever with one hand.

Further, in the endoscope described in JP2011-072455A, in a case where the user releases his/her hand from the elevating operation lever, the elevating operation lever is temporarily stopped by the friction resistance between the friction resistance applying spring material and the fixed wall. However, in a case where a treatment is performed, in the case of a treatment tool having high rigidity such as a stent or a puncture needle, a reaction force received from the treatment tool is large, so that the stopped state cannot be held only by the friction resistance of the friction resistance applying spring material. Therefore, the treatment cannot be stably performed in a case where the elevator is at a position in the middle of the elevated position and the fallen position.

The present invention has been made in view of the above circumstances, and an object of the present invention is to provide an endoscope that can be easily operated with only one hand in a case of changing a lead-out direction of a treatment tool, and with which various treatments can be performed in a state where the lead-out direction is held.

An endoscope according to an aspect of the present invention comprises an insertion part, an operation part, a distal end portion body, an elevator, an elevating operation lever, and a locking mechanism. The insertion part is to be inserted into a subject. The operation part is provided at a proximal end of the insertion part. The distal end portion body is located at a distal end of the insertion part and communicates with a treatment tool lead-out port. The elevator is provided at the distal end portion body, causes a treatment tool led out of the treatment tool lead-out port to elevate, and is provided to be movable between an elevated position and a fallen position. The elevating operation lever causes the elevator to move to the elevated position in accordance with rotation in a first direction and causes the elevator to move to the fallen position in accordance with rotation in a second direction. The locking mechanism brings the elevating operation lever into a locked state. The locking mechanism includes a lock member and a biasing member, and in a case where the elevating operation lever is not operated, the lock member is biased to a lock position by a biasing force and the elevating operation lever is in the locked state, and in a case where the elevating operation lever is operated, the locked state of the elevating operation lever is released by a lock release operation against the biasing force. The lock member is provided in the elevating operation lever. The biasing member applies the biasing force to the lock member.

It is preferable that the locking mechanism includes a locking piece that is provided in the operation part, a locked member that serves as the lock member, the locked member being slidably attached to the elevating operation lever and being biased by the biasing force to a locked position where the locked member is locked to the locking piece, and a pressing operation member that is provided integrally with the locked member, and in a case where the pressing operation member is not pressed, the locked member is locked to the locking piece, and in a case where the elevating operation lever is operated, the pressing operation member is pressed against the biasing force to be moved integrally with the locked member and the locked member is moved from the locked position to a locked release position.

It is preferable that the locking mechanism includes a fitting groove that is provided in the operation part, and a fitting member that constitutes a part of the elevating operation lever and is biased by the biasing force of the biasing member to a fitting position where the fitting member is fitted to the fitting groove, the fitting member being slidably attached to a body of the elevating operation lever, and in a case where the fitting member is not pressed, the fitting member is fitted to the fitting groove, and in a case where the elevating operation lever is operated, the fitting member is pressed and moved against the biasing force and the fitting member is moved from the fitting position to a fitting release position.

It is preferable that the fitting member includes a fitting projection that extends in a direction parallel to a central axis of the elevating operation lever, and the fitting groove is an arc-shaped groove that is fitted to the fitting projection and is formed around the central axis.

It is preferable that the fitting member includes a fitting projection that protrudes in a radial direction orthogonal to a central axis of the elevating operation lever, and the fitting groove is a plurality of grooves that are fitted to the fitting projection and are arranged around the central axis.

It is preferable that at least some components are disposable.

According to the present invention, in a case where the lead-out direction of the treatment tool is changed, the operation can be easily performed with only one hand, and various treatments can be performed in a state where the lead-out direction is held.

### BRIEF DESCRIPTION OF THE DRAWINGS

FIG. 1 is a schematic diagram illustrating a configuration of an endoscope system.

FIG. 2 is an external view of an endoscope and a treatment tool.

FIG. 3 is a perspective view showing a distal end portion of the endoscope.

FIG. 4 is an exploded perspective view of the distal end portion of the endoscope.

FIG. 5 is a perspective view of an operation part of the endoscope.

FIG. 6 is a plan view of a treatment tool elevating mechanism.

FIG. 7 is an exploded perspective view of the treatment tool elevating mechanism.

FIG. 8 is a cross-sectional view of main parts of an operation part.

FIG. 9 is a perspective view showing a configuration of a locked member and a locking piece.

FIG. 10 is a cross-sectional view of main parts around the elevating operation lever and a locking mechanism.

FIGS. 11A and 11B are explanatory views illustrating an operation of the locking mechanism, showing a locked state (FIG. 11A) and an unlocked state (FIG. 11B).

FIGS. 12A and 12B are explanatory views illustrating the operation of the locking mechanism, illustrating a state (FIG. 12A) in which the elevating operation lever is rotated from unlocking and a locked state (FIG. 12B) after the elevating operation lever is rotated.

FIG. 13 is a modified example in which the locking piece of the locking mechanism is formed in a gear shape.

FIG. 14 is a modified example in which the locking mechanism is configured by a plurality of locking pieces arranged in an arc shape.

FIG. 15 is a cross-sectional view of the main parts of the operation part in a second embodiment, and is a cross-sectional view of the main parts in a case where the locking mechanism is in the locked state.

FIG. 16 is a perspective view of the operation part in the second embodiment.

FIG. 17 is a cross-sectional view of the main parts of the operation part in the second embodiment, and is a cross-sectional view of the main parts in a case where the locking mechanism is in the unlocked state.

FIG. 18 is a cross-sectional view of the main parts of the operation part in a third embodiment, and is a cross-sectional view of the main parts in a case where the locking mechanism is in the locked state.

FIG. 19 is a perspective view of the operation part in the third embodiment.

FIG. 20 is an explanatory view illustrating the operation of the locking mechanism in the third embodiment.

FIG. 21 is a cross-sectional view of the main parts of the operation part in a fourth embodiment, and is a cross-sectional view of the main parts in a case where the locking mechanism is in the locked state.

FIG. 22 is a perspective view of the operation part in the fourth embodiment.

### DESCRIPTION OF THE PREFERRED EMBODIMENTS

### First Embodiment

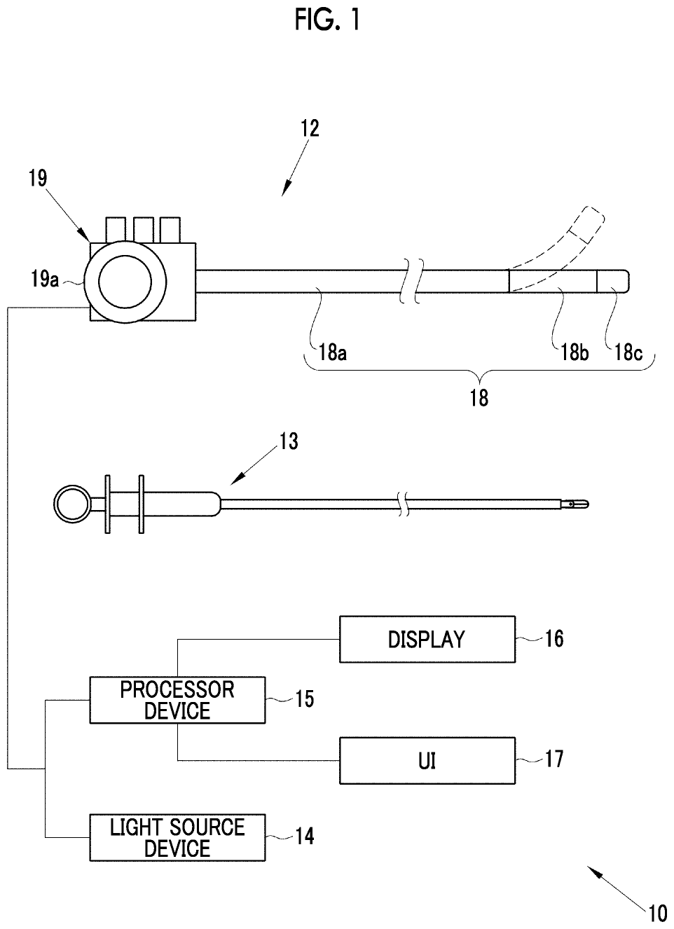

As shown in FIG. 1, an endoscope system 10 comprises an endoscope 12, a treatment tool 13, a light source device 14, a processor device 15, a display 16, and a user interface (UI) 17. The endoscope 12 images an observation target. The light source device 14 emits illumination light with which the observation target is irradiated. The processor device 15 performs system control of the endoscope system 10. The display 16 is a display unit that displays an observation image or the like based on an endoscope image. The UI 17 includes a keyboard, a mouse, a touch pad, a microphone, and the like, and receives an input operation of a doctor who is a user.

It is preferable that at least some of components forming the endoscope 12 are formed of a resin material, a rubber material, a metal material, or the like and are discarded as disposables. In addition, in a case of the metal material, it is more preferable that the components are formed by metal injection molding.

The endoscope 12 is optically connected to the light source device 14 and is electrically connected to the processor device 15. The endoscope 12 has an insertion part 18 to be inserted into a subject and an operation part 19 provided at a proximal end of the insertion part 18. The insertion part 18 includes a soft portion 18a, a bendable portion 18b, and a distal end portion 18c which are consecutively installed in this order from the proximal end toward a distal end. The bendable portion 18b is bent by operating an angle knob 19a of the operation part 19. As a result, the distal end portion 18c faces in a desired direction.

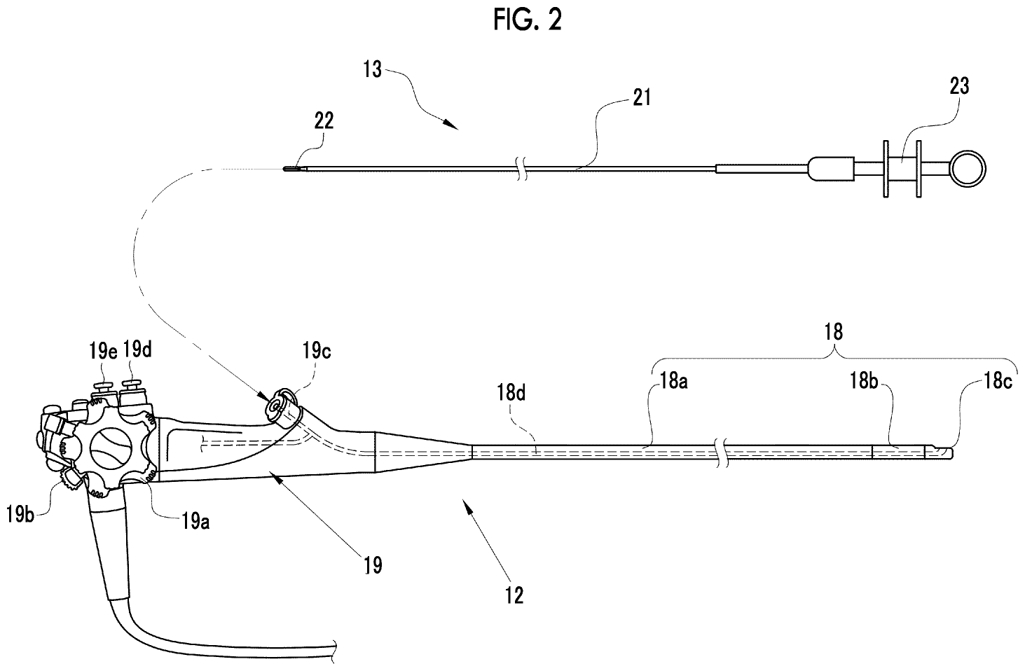

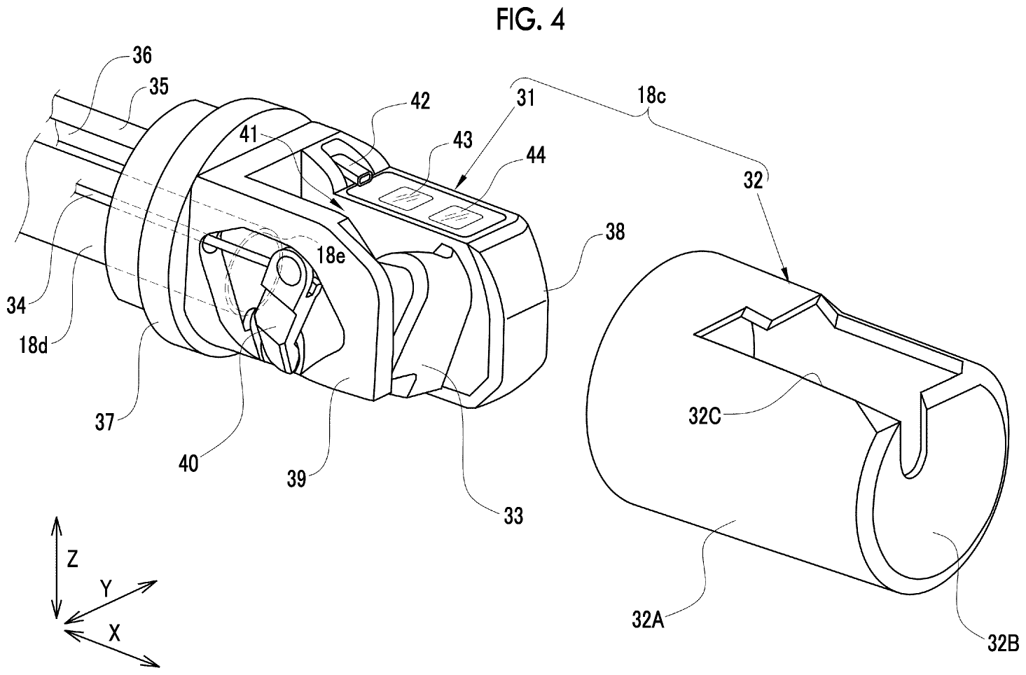

As shown in FIG. 2, the operation part 19 is provided with an elevating operation lever 19b, a treatment tool lead-in port 19c, an air and water supply button 19d, and a suction button 19e, in addition to the angle knob 19a. The treatment tool lead-in port 19c is an entrance for inserting the treatment tool 13. The treatment tool 13 inserted into the treatment tool lead-in port 19c is guided to the elevator housing portion 41 (see FIG. 4) of the distal end portion 18c.

By operating the elevating operation lever 19b, a treatment tool elevating mechanism 45 to be described later operates, and an elevator 33 rotates. In a case where the elevator 33 rotates, an advancing direction of the treatment tool 13 guided to the elevator housing portion 41 is bent, so that the treatment tool 13 is guided in a direction toward an opening window 32C on an upper surface side of the elevator housing portion 41, and is led out of the opening window 32C.

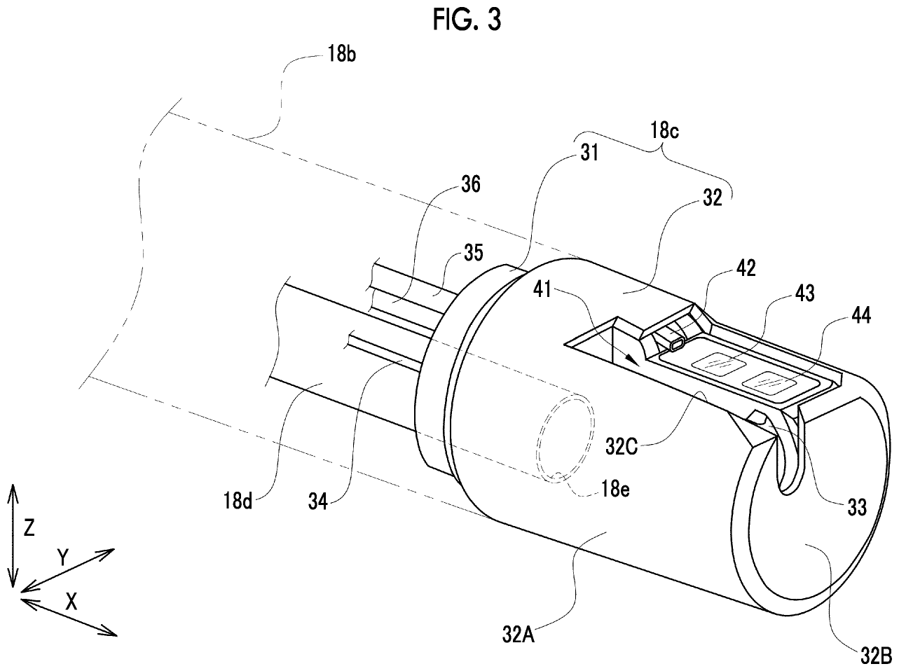

In a case where the air and water supply button 19d is operated, air and water are supplied to an air and water supply tube (not shown) and the air and water is jetted from an air and water supply nozzle 42 (see FIGS. 3 and 4) provided in the distal end portion body 31. Further, in a case where the suction button 19e is operated, body fluids, such as blood, can be suctioned via the treatment tool channel 18d from a suction port that also serves as the treatment tool lead-out port 18e (see FIGS. 3 and 4) disposed in the distal end portion body 31.

An image sensor 43, an illumination optical system 44, and the like, which will be described later, are provided in the distal end portion 18c. The image sensor 43 is preferably a charge coupled device (CCD) image sensor, a complementary metal-oxide semiconductor (CMOS) image sensor, or the like.

The processor device 15 is electrically connected to the display 16 and the UI 17. The processor device 15 performs image processing or the like on the endoscope image captured by the image sensor 43 and displays the image-processed endoscopic image on the display 16.

The treatment tool channel 18d for inserting the treatment tool 13 is disposed in the insertion part 18. One end of the treatment tool channel 18d is connected to the distal end portion body 31, and the other end of the treatment tool channel 18d is connected to the treatment tool lead-in port 19c provided in the operation part 19.

The treatment tool 13 is a treatment tool for an endoscope that is inserted into the subject together with the insertion part 18 through the treatment tool channel 18d. As the treatment tool 13, for example, a biopsy forcep, a snare, a stent, a puncture needle, a high-frequency treatment tool, an ultrasonic treatment tool, and the like is combined with the endoscope 12.

The treatment tool 13 comprises a flexible sheath 21, an operation wire (not shown), a distal end portion 22, and an operation part 23. The flexible sheath 21 is a tubular sheath formed of a flexible material, for example, a soft resin, and is inserted into the treatment tool channel 18d of the endoscope 12. The operation wire is provided integrally with the distal end portion 22, and is inserted in the flexible sheath 21.

As shown in FIGS. 3 and 4, the distal end portion 18c includes a distal end portion body 31 and a cap 32, and is formed by mounting the cap 32 on the distal end portion body 31. A configuration of the distal end portion 18c is not limited thereto, and the distal end portion body 31 and the cap 32 may be integrally fixed to each other so as not to be detached by the user. The distal end portion body 31 is provided on a distal end side of the insertion part 18 (see FIG. 1), and the elevator 33 is provided in the distal end portion body 31.

The endoscope 12 is, for example, a side-viewing endoscope used as a duodenoscope, and the distal end portion 18c shown in FIGS. 3 and 4 has a configuration of the side-viewing endoscope. FIGS. 3 and 4 show the treatment tool channel 18d, an elevating operation wire 34, a signal cable 35, and a light guide 36 which are disposed inside the insertion part 18 of the endoscope 12. The treatment tool channel 18d guides the distal end portion of the treatment tool 13 to the distal end portion body 31. The elevating operation wire 34 is an operation wire for performing an operation of rotating the elevator 33. Illustrations of an air and water supply channel and the like connected to the air and water supply nozzle 42 are omitted to prevent complication.

The cap 32 is formed in a substantially tubular shape of which a distal end side is sealed, and has a peripheral surface portion 32A and an end surface portion 32B. The opening window 32C having a substantially rectangular shape is formed in a part of the peripheral surface portion 32A. In the example shown in FIGS. 3 and 4, the opening window 32C is an opening portion cut out from the peripheral surface portion 32A to the end surface portion 32B. Hereinafter, descriptions will be made with a central axis direction of the distal end portion body 31 and the cap 32 as an X-axis direction, a vertical direction orthogonal to the X-axis direction as a Z-axis direction, and a horizontal direction orthogonal to the X-axis direction and the Z-axis direction as a Y-axis direction.

In a case where the cap 32 is mounted on the distal end portion body 31, the cap 32 covers the elevator housing portion 41 to be described later, and the opening window 32C is opened toward the Z-axis direction. Accordingly, the treatment tool lead-out port 18e of the treatment tool channel 18d communicates with the opening window 32C through the elevator housing portion 41. The image sensor 43 and the illumination optical system 44 are exposed through the opening window 32C. The cap 32 is coaxially mounted on the distal end portion body 31.

The cap 32 is made of an elastic material, for example, a rubber material such as fluororubber or silicon rubber, or a resin material such as polysulfone or polycarbonate. The configuration of the distal end portion 18c is not limited thereto, and the distal end portion body 31 and the cap 32 may be integrally fixed to each other so as not to be detached by the user. A protruding engaging portion (not shown) to be engaged with a groove-like engaged portion (not shown) formed on the distal end portion body 31 is provided on a proximal end side of the cap 32, and the engaging portion is engaged with the engaged portion, so that the cap 32 is attachably and detachably mounted on the distal end portion body 31.

As shown in FIG. 4, the distal end portion body 31 includes a disk portion 37 and a pair of partition wall portions 38 and 39. The distal end portion body 31 is made of, for example, a resin material. The pair of partition wall portions 38 and 39 are provided to protrude from the disk portion 37 in the X-axis direction. The partition wall portions 38 and 39 are disposed opposite each other in the Y-axis direction. Further, the elevator housing portion 41 housing the elevator 33 is provided between the partition wall portion 38 and the partition wall portion 39. The elevator housing portion 41 is opened in the Z-axis direction.

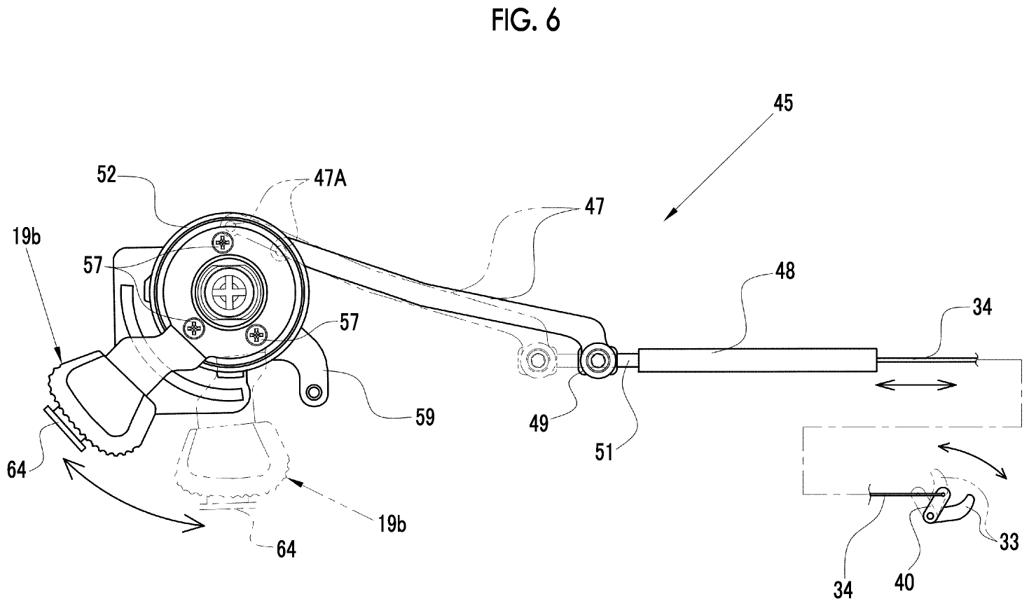

The elevator housing portion 41 communicates with the treatment tool lead-out port 18e of the treatment tool channel 18d. The elevator 33 causes the treatment tool 13 led out of the treatment tool lead-out port 18e to elevate. The elevator 33 is rotatably attached inside the elevator housing portion 41 via a rotational shaft member 40 (see FIGS. 4 and 6), and is movable between an elevated position (a position indicated by a two dot chain line) and a fallen position (a position indicated by a solid line). A distal end of an elevating operation wire 34 is connected to an end portion of the rotational shaft member 40. As the elevating operation wire 34 is pushed or pulled, the elevator 33 rotates from the fallen position to the elevated position. Accordingly, the lead-out direction of the distal end portion 22 of the treatment tool 13 led out to the treatment tool lead-out port 18e can be changed.

The disk portion 37 is coupled to a distal end side of the bendable portion 18b. The bendable portion 18b is configured by covering an outer periphery of a structure in which a plurality of bendable pieces are rotatably connected with a tubular net body, a rubber outer skin, or the like. The disk portion 37 is fixed to the bendable piece positioned on the most distal end side among the plurality of bendable pieces forming the bendable portion 18b by, for example, screwing or adhesion using an adhesive.

The partition wall portion 38 is disposed adjacent to the elevator housing portion 41 in the Y-axis direction. The partition wall portion 38 comprises the air and water supply nozzle 42, the image sensor 43, and the illumination optical system 44. The image sensor 43 is electrically connected to the signal cable 35, and the illumination optical system 44 is optically connected to the light guide 36. The air and water supply nozzle 42 is provided in the distal end portion body 31 toward the image sensor 43 and the illumination optical system 44, and accordingly, the image sensor 43 and the illumination optical system 44 are cleaned by air and water jetted from the air and water supply nozzle 42.

The signal cable 35 and the light guide 36 are respectively connected to the processor device 15 and the light source device 14 through the insertion part 18, the operation part 19, a connector (not shown), and the like. The processor device 15 performs image processing or the like on the imaging signal acquired by the image sensor 43 and causes the display 16 to display the observation image. The light guide 36 is formed of an optical fiber cable or the like, transmits the illumination light emitted from the light source device 14, and irradiates the observation target with the illumination light through the illumination optical system 44.

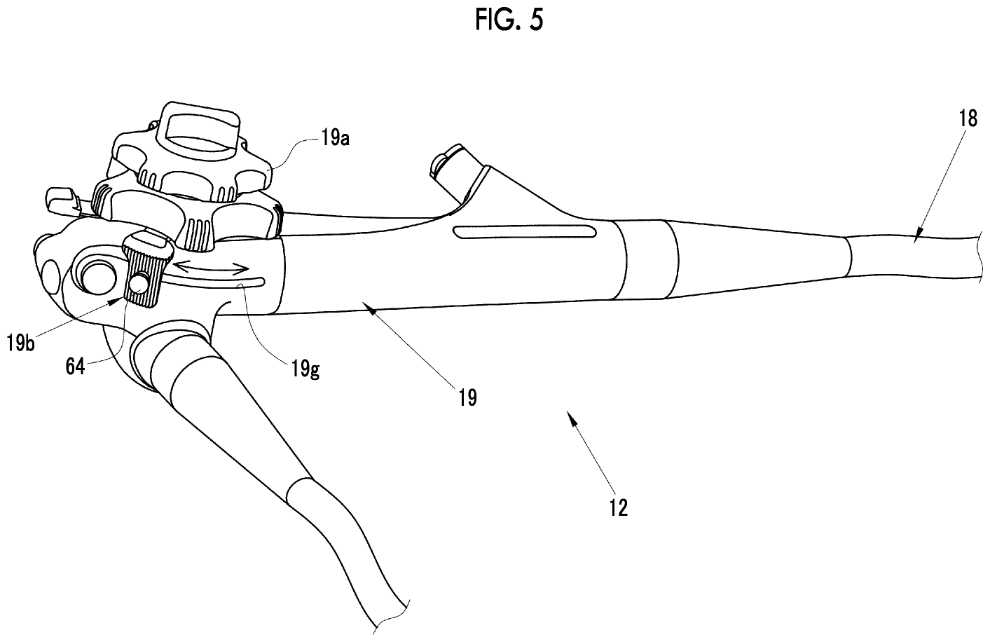

As shown in FIG. 5, the elevating operation lever 19b is provided with a lock release button 64. The elevating operation lever 19b is in a locked state by a locking mechanism 61 to be described later, and the locked state is released by pressing the lock release button 64 in a case where the elevating operation lever 19b is operated. The lock release button 64 corresponds to a pressing operation member in the claims.

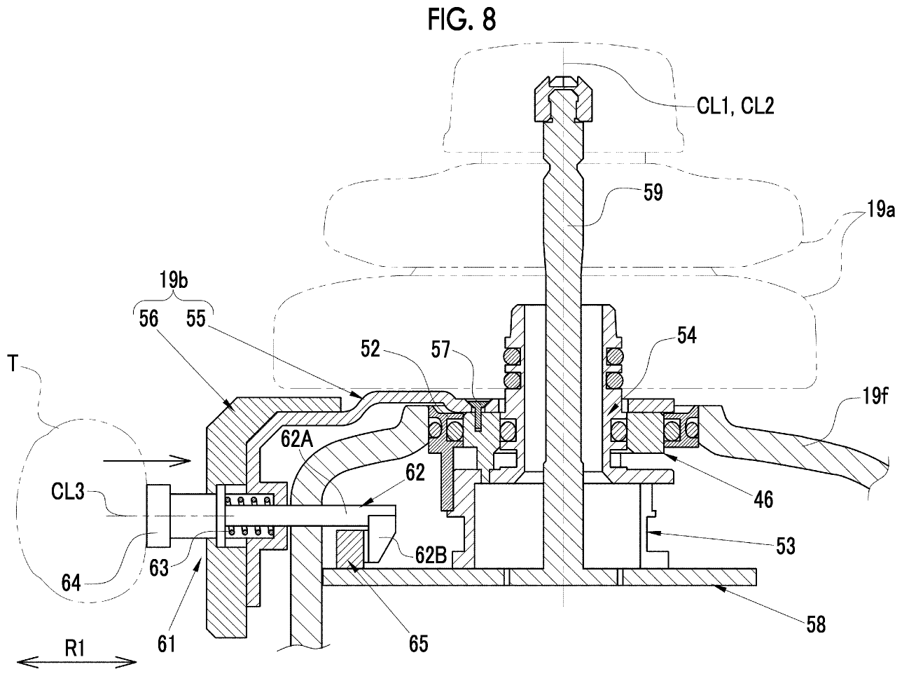

As shown in FIG. 6, the treatment tool elevating mechanism 45 performs an operation of pushing or pulling the elevating operation wire 34 according to a rotation operation of the elevating operation lever 19b. In a case where the elevating operation wire 34 is pushed or pulled, the above-described rotational shaft member 40 and the elevator 33 rotate. The elevating operation lever 19b is operated by a thumb T (see FIG. 8).

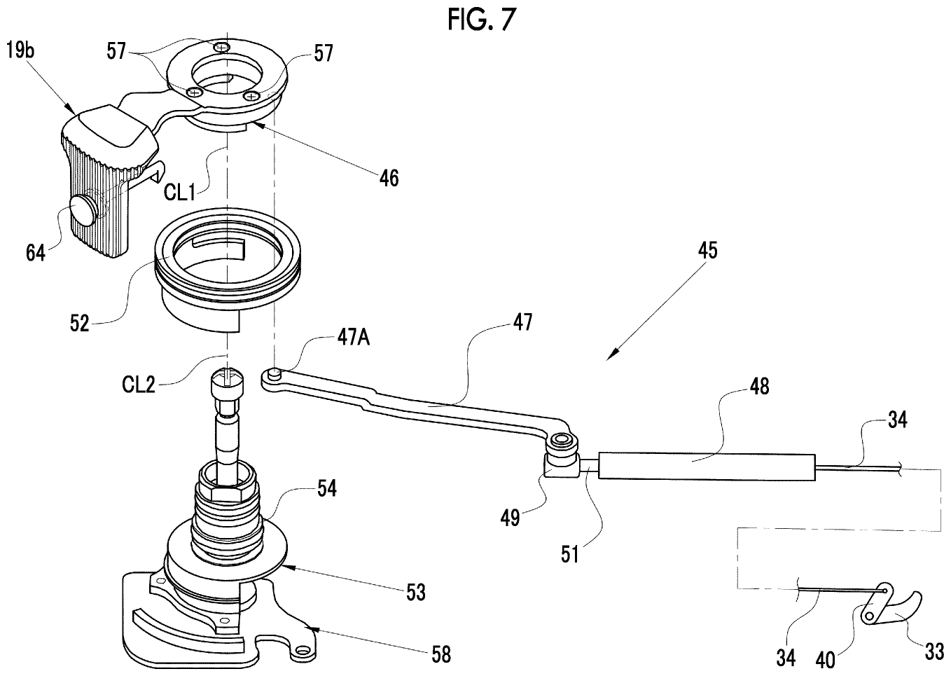

As shown in FIG. 7, the treatment tool elevating mechanism 45 comprises the elevating operation lever 19b, a rotary ring 46, a crank member 47, a guide barrel 48, a connection head 49, a slider 51, a fixed ring 52, and a bearing member 53. The rotary ring 46 is formed in a cylindrical shape.

The bearing member 53 is provided with a rotational shaft 54. The rotary ring 46 is coaxially and rotatably attached to the rotational shaft 54. That is, a central axis CL1 of the rotary ring 46 and a central axis CL2 of the rotational shaft 54 coincide with each other. The bearing member 53 and the fixed ring 52 hold the rotary ring 46. The fixed ring 52 supports an outer peripheral surface of the rotary ring 46 and is fixed to a case 19f (see FIG. 8) constituting an exterior of the operation part 19. The fixed ring 52 prevents the rotary ring 46 from falling off from the operation part 19.

As shown in FIG. 8, the elevating operation lever 19b includes a plate member 55 and a finger hook member 56. The plate member 55 is formed of, for example, a metal plate, and is bent in a substantially L shape. One end of the plate member 55 is formed in an annular shape and is connected to the rotary ring 46 by, for example, screwing with a screw 57. The finger hook member 56 is formed of, for example, a resin material, and covers the other end of the plate member 55. The plate member 55 and the finger hook member 56 are integrally fixed to each other by, for example, adhesion or screwing. The finger hook member 56 is subjected to unevenness processing for forming unevenness on a front surface side on which a user hooks a finger. As the unevenness formed on the finger hook member 56, for example, a plurality of rows of thin grooves are formed. Thus, in a case where the user hooks his/her finger on the finger hook member 56, the finger does not easily slip.

One end of the crank member 47 is rotatably connected to the elevating operation lever 19b via a connection pin 47A, and the other end of the crank member 47 is connected to the connection head 49. The connection head 49 is attached to one end of the slider 51, and the elevating operation wire 34 is connected to the other end of the slider 51. The slider 51 is slidably supported by the guide barrel 48. The guide barrel 48 is fixed to the case 19f.

The bearing member 53 is fixed to the case 19f via a frame member 58. The rotational shaft 54 is formed hollow. An attachment shaft 59 of the angle knob 19a is inserted into and fixed to the rotational shaft 54. Accordingly, the angle knob 19a is attached to the rotational shaft 54 via the attachment shaft 59. In FIG. 8, an internal mechanism of the angle knob 19a, a mechanism for bending the bendable portion 18b, and the like are omitted in order to prevent complication.

The crank member 47 and the slider 51 convert the rotation by the operation of the elevating operation lever 19b into a linear motion of the elevating operation wire 34, that is, a push or pull operation. The elevating operation lever 19b causes the elevator 33 to move to the elevated position along with the rotation in a counterclockwise direction (a first direction) and causes the elevator 33 to move to the fallen position along with the rotation in a clockwise direction (a second direction), by the push or pull operation of the treatment tool elevating mechanism 45.

The locking mechanism 61 includes a locked member 62, a spring member 63, the lock release button 64, and a locking piece 65. The locking piece 65 is fixed to the frame member 58. The locked member 62 corresponds to a lock member in the claims. Hereinafter, descriptions will be made with a direction parallel to a central axis CL3 of the lock release button 64 and orthogonal to the central axes CL1 and CL2 as a radial direction R1 (see FIG. 8).

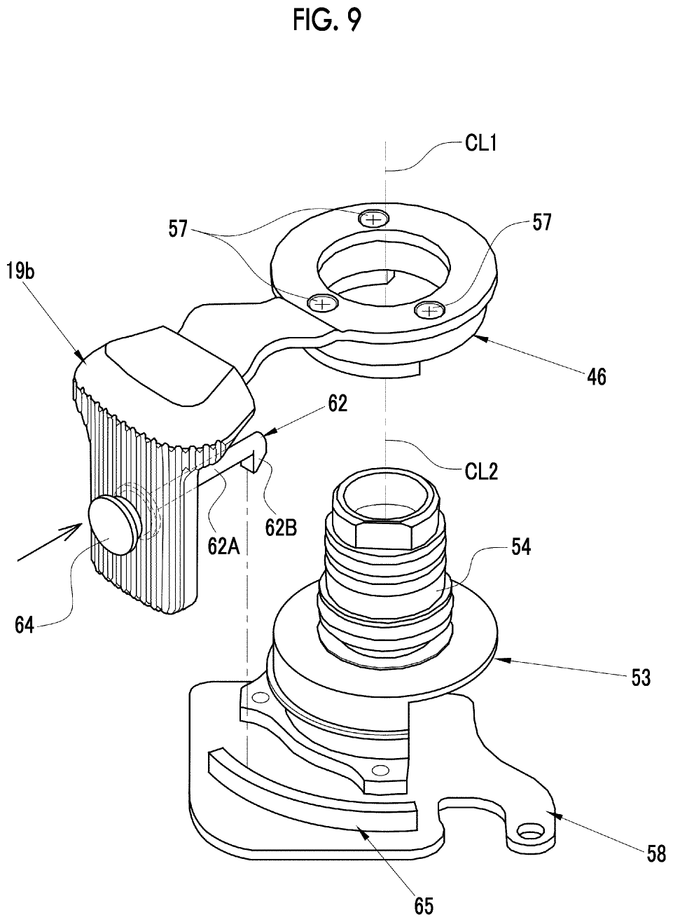

As shown in FIG. 9, the locking piece 65 is an arc-shaped projection portion that is provided coaxially with the rotational shaft 54, that is, around the central axis CL2. The locking piece 65 is provided inside the case 19f via the frame member 58. In FIG. 9, illustrations of the spring member 63 and the like are omitted in order to avoid complication.

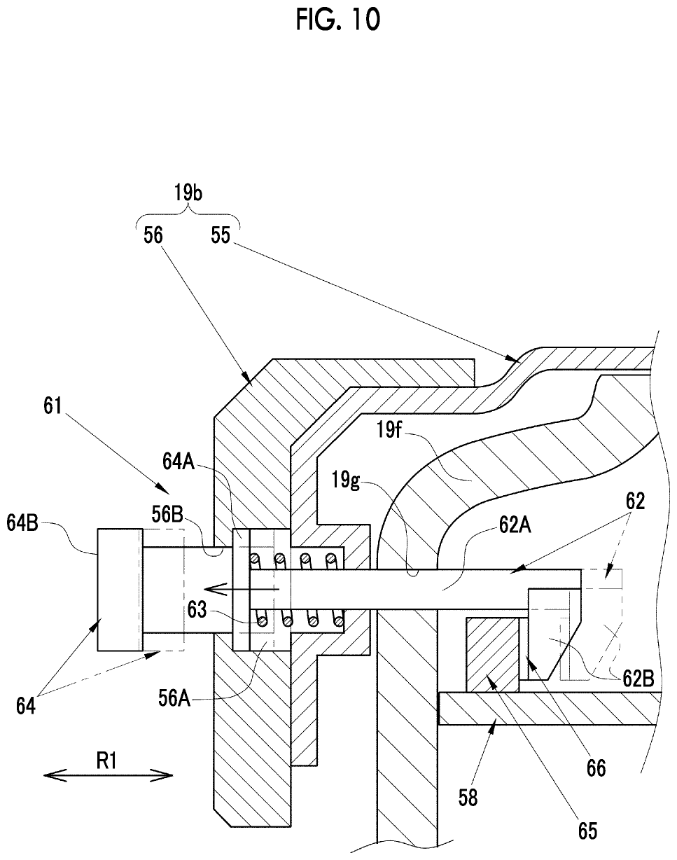

As shown in FIG. 10, the locked member 62 is provided integrally with the lock release button 64. The locked member 62 has a columnar portion 62A formed in a columnar shape and a locked claw 62B formed at one end of the columnar portion 62A. The lock release button 64 is installed consecutively to the other end of the columnar portion 62A. The columnar portion 62A is attached to the elevating operation lever 19b so as to be slidable along the radial direction R1. A groove 19g (see FIG. 5) is formed on the case 19f. The columnar portion 62A penetrates the groove 19g and enters the inside of the case 19f. With the rotation of the elevating operation lever 19b, the columnar portion 62A rotates along the groove 19g.

A storage portion 56A and a through-hole 56B into which a lock release button 64 is inserted are formed in the finger hook member 56. The storage portion 56A stores the spring member 63 and a retaining portion 64A of the lock release button 64 to be described later. As the spring member 63, for example, a coil spring is used. The spring member 63 is externally fitted to an outer peripheral surface of the columnar portion 62A. The lock release button 64 is formed in a substantially columnar shape, and has the retaining portion 64A at one end where the locked member 62 is consecutively installed and a pressing surface 64B at the other end. The retaining portion 64A is a projection portion that protrudes from an end portion. The retaining portion 64A is locked to a peripheral edge of the through-hole 56B.

The spring member 63 is stored in the storage portion 56A in a state where one end of the spring member 63 is in contact with the lock release button 64 and the other end thereof is in contact with the plate member 55. As a result, the spring member 63 applies a biasing force to the locked member 62 together with the lock release button 64. Since the retaining portion 64A is locked to the peripheral edge of the through-hole 56B, the lock release button 64 and the spring member 63 are restricted from being separated from the elevating operation lever 19b.

In a case where the elevating operation lever 19b is not operated, that is, in a case where the lock release button 64 is not pressed, the locked member 62 is biased toward the outside of the radial direction R1 by the biasing force of the spring member 63. Therefore, the locked claw 62B of the locked member 62 is brought into contact with the locking piece 65. Since the locked claw 62B is locked to the locking piece 65, the elevating operation lever 19b is in the locked state. Hereinafter, a position where the locked claw 62B is locked to the locking piece 65 is referred to as a locked position or a lock position (a position indicated by a solid line).

In the present embodiment, a friction member 66 is provided on the locked claw 62B such that the locking piece 65 reliably locks the locked claw 62B to bring the elevating operation lever 19b into the locked state. The friction member 66 is disposed at a position facing the locking piece 65, so that the friction member 66 is brought into contact with the locking piece 65 and a frictional force with the locking piece 65 is generated. The locked member 62 is reliably locked by the biasing force of the spring member 63 and the frictional force of the friction member 66, and the rotation thereof is restricted. Accordingly, the elevating operation lever 19b is brought into the locked state. The friction member 66 is made of, for example, rubber or a soft resin.

On the other hand, in a case where the elevating operation lever 19b is operated, the lock release button 64 is pressed as a lock release operation. In a case where the lock release button 64 is pressed against the biasing force of the spring member 63, that is, toward the inside of the radial direction R1, the lock release button 64 is moved integrally with the locked member 62, and the locked member 62 is moved to the inside of the radial direction R1. Therefore, the locked claw 62B is separated from the locking piece 65. Since the locked claw 62B is released from the locking piece 65, the locked state of the elevating operation lever 19b is released. Hereinafter, a position where the locked claw 62B is released from the locking piece 65 is referred to as a locked release position or a lock release position (a position indicated by a two dot chain line).

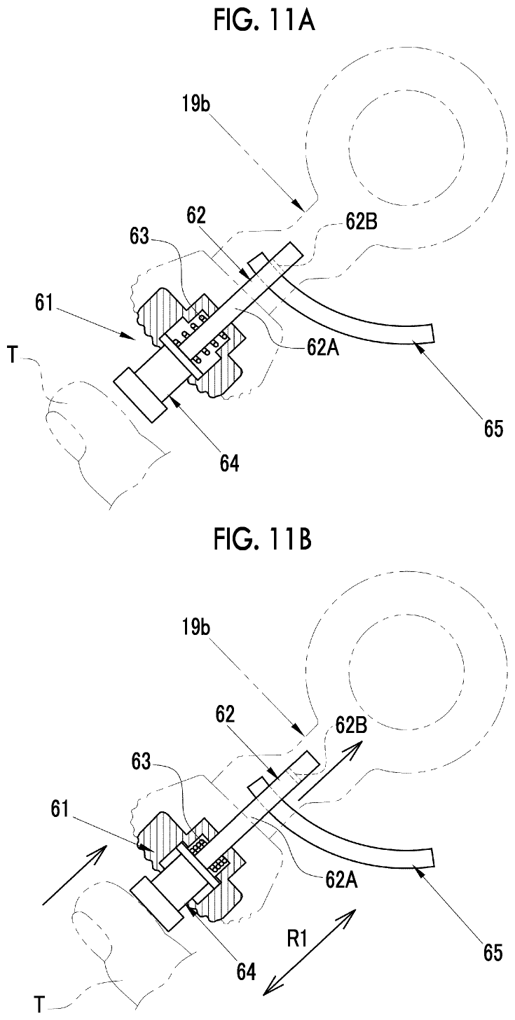

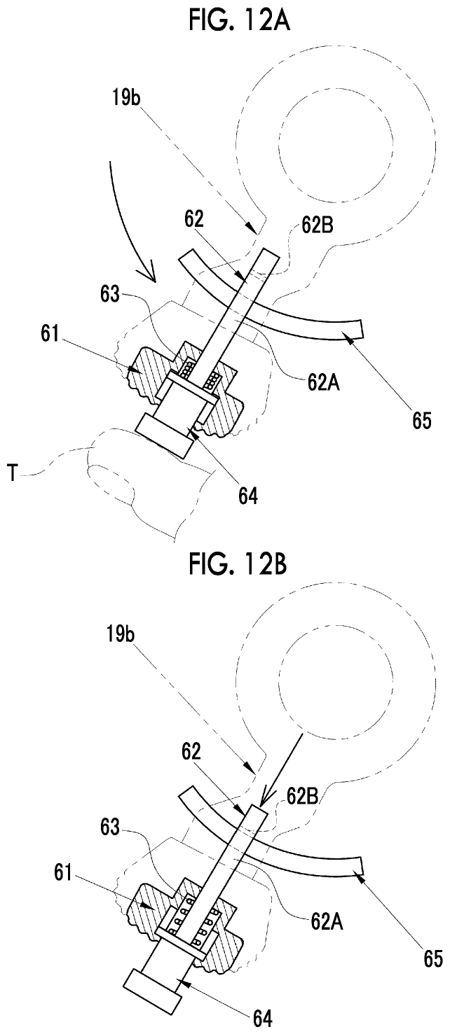

With reference to FIGS. 11A to 12B, an operation in a case where the doctor who is the user inserts the treatment tool 13 into the treatment tool channel 18d of the endoscope 12 and performs a treatment on a patient who is the subject will be described. The doctor inserts the insertion part 18 of the endoscope 12 into a body of the patient. Further, the treatment tool 13 is inserted into the treatment tool channel 18d through the treatment tool lead-in port 19c. The treatment tool 13 inserted into the treatment tool channel 18d is led out of the treatment tool lead-out port 18e into the subject.

As shown in FIG. 11A, in a case where the elevating operation lever 19b is not operated, the locked claw 62B is located at the locked position and is locked to the locking piece 65, so that the elevating operation lever 19b is in the locked state. In a case where the elevator 33 is at the fallen position, the treatment tool 13 is not elevated at all. In this case, the treatment tool 13 is not present in an observation range imaged by the image sensor 43. For example, the doctor rotationally operates the elevating operation lever 19b to move the elevator 33 from the fallen position to the elevated position in order to make the treatment tool 13 enter the observation range.

As shown in FIG. 11B, in a case where the elevating operation lever is operated, the lock release button 64 is pressed inward in the radial direction R1 by the thumb T of the user as the lock release operation. The locked member 62 is moved to the locked release position against the biasing force of the spring member 63, and the locked claw 62B is separated from the locking piece 65. Since the locked claw 62B is released from the locking piece 65, the locked state of the elevating operation lever 19b is released.

As shown in FIG. 12A, the elevating operation lever 19b released from the locked state becomes rotatable. The doctor can rotate the elevator 33 in a desired direction by holding a state where the lock release button 64 is pressed by the thumb T and rotationally operating the elevating operation lever 19b without separating the thumb T. For example, the doctor determines that the lead-out direction of the treatment tool 13 is changed to a desired direction in a case where the distal end portion 22 of the treatment tool 13 enters the observation range imaged by the image sensor 43.

In a case where the doctor changes the lead-out direction of the treatment tool 13 in the desired direction, the elevator 33 may be at a position in the middle of the fallen position and the elevated position. In this case, as shown in FIG. 12B, the doctor releases the thumb T pressing the lock release button 64, and stops the rotation operation of the elevating operation lever 19b. The locked member 62 returns to the locked position from the locked release position by the biasing force of the spring member 63. Since the locked member 62 is locked to the locking piece 65, the elevating operation lever 19b is in the locked state. Since the elevating operation lever 19b is locked, the doctor can perform a treatment in a state where the lead-out direction of the treatment tool 13 is held in the desired direction. In a state where the lead-out direction is held in the desired direction, the distal end portion 22 of the treatment tool 13 is led out of the opening window 32C, whereby the doctor can perform various treatments, such as resection and collection of an observation site.

As described above, in a case of changing the lead-out direction of the treatment tool 13, the doctor can perform the lock release and the rotation operation by the elevating operation lever 19b with one hand. Then, various treatments can be performed in a state where the lead-out direction of the treatment tool 13 is held in the desired direction.

Further, in a case where the lock release button 64 is not pressed, the locked member 62 is locked to the locking piece, and in a case where the elevating operation lever 19b is operated, the lock release button 64 is pressed against the biasing force of the spring member 63 to be moved integrally with the locked member 62, so that the locked member 62 is moved from the locked position to the locked release position. Accordingly, even though the elevator 33 is in the middle of the elevated position and the fallen position, the locking mechanism 61 operates, and the elevating operation lever 19b can be reliably stopped.

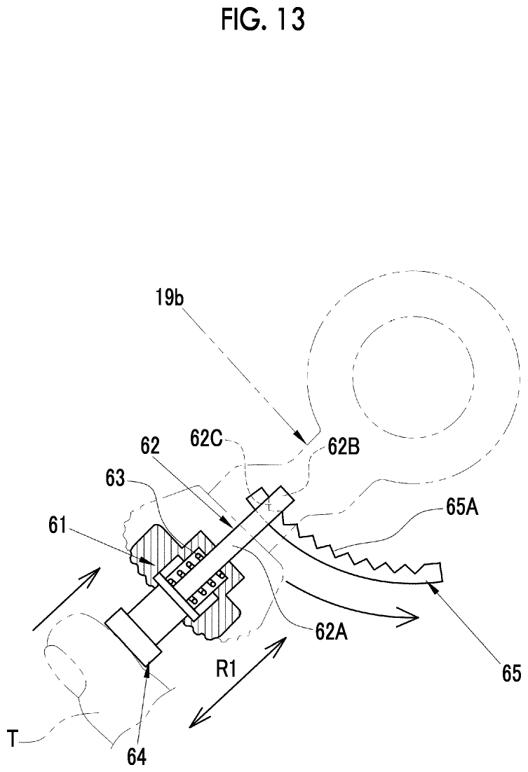

In the first embodiment, in order for the locking piece 65 to reliably lock the locked claw 62B, the friction member 66 is provided on the locked claw 62B, but the present invention is not limited thereto, and it is sufficient that the locking piece 65 is configured to reliably lock the locked claw 62B. For example, in a modified example shown in FIG. 13, a protrusion 62C is formed on the locked claw 62B, and a plurality of recesses 65A are formed in the locking piece 65. In this case, the protrusion 62C is a mountain-shaped protrusion which is disposed at the position facing the locking piece 65 and protrudes toward the locking piece 65. The locking piece 65 is formed in a gear shape composed of the plurality of recesses 65A matched with the protrusion 62C. In a case where the lock release button 64 is not pressed, the locked member 62 is biased toward the outside of the radial direction R1 by the biasing force of the spring member 63. Therefore, the protrusion 62C is brought into contact with any one of the recesses 65A. Since the locked claw 62B is locked by the locking piece 65, the elevating operation lever 19b is in the locked state.

On the other hand, as the lock release button 64 is pressed against the biasing force of the spring member 63, that is, toward the inside in the radial direction R1, the locked member 62 is moved to the inside in the radial direction R1. Therefore, the protrusion 62C is separated from the recess 65A. Since the locked claw 62B is released from the locking piece 65, the locked state of the elevating operation lever 19b is released. As described above, even in a case where the elevator 33 is in the middle of the elevated position and the fallen position, the locking mechanism 61 operates, and the elevating operation lever 19b can be reliably stopped. Accordingly, the same effects as those of the first embodiment can be obtained.

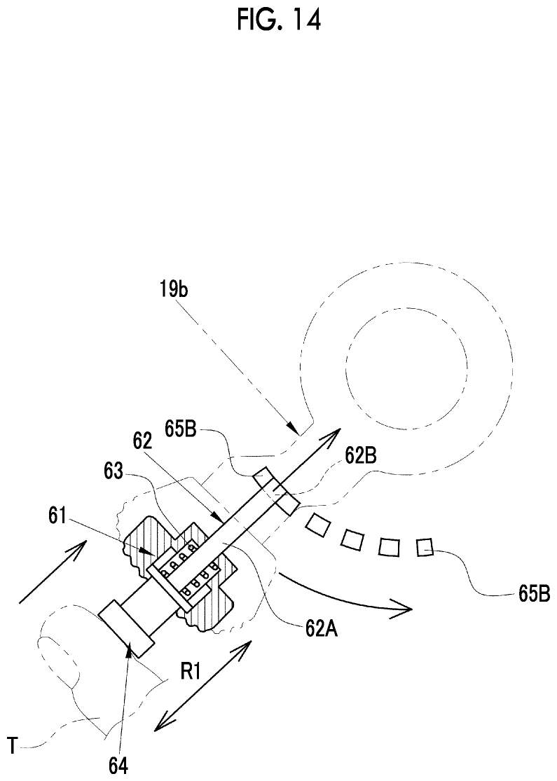

Further, as in a modified example shown in FIG. 14, a plurality of locking pieces 65B may be arranged in an arc shape. In this case, the plurality of locking pieces 65B are arranged along an arc located around the central axis CL1. An interval between the adjacent locking pieces 65B is formed in accordance with a width of the locked claw 62B. In a case where the lock release button 64 is not pressed, the locked member 62 is biased toward the outside of the radial direction R1 by the biasing force of the spring member 63. Therefore, the locked claw 62B enters between the adjacent locking pieces 65B. Since the locked claw 62B is locked by the locking piece 65, the elevating operation lever 19b is in the locked state.

On the other hand, as the lock release button 64 is pressed against the biasing force of the spring member 63, that is, toward the inside in the radial direction R1, the locked member 62 is moved to the inside in the radial direction R1. Therefore, the locked claw 62B is separated from between the adjacent locking pieces 65B. Since the locked claw 62B is released from the locking piece 65, the locked state of the elevating operation lever 19b is released. Accordingly, the same effects as those of the first embodiment can be obtained.

### Second Embodiment

In the first embodiment, the endoscope comprises the locking mechanism 61 in which the locked member 62 is biased to the locked position to bring the elevating operation lever 19b into the locked state, and the lock release button 64 is pressed against the biasing force to be moved the locked member 62 to the locked release position to release the locked state of the elevating operation lever 19b, but the present invention is not limited thereto. In the second embodiment described below, an endoscope that comprises a locking mechanism in which a fitting member is fitted to a fitting groove to bring an elevating operation lever into the locked state, and the fitting member is moved from a fitting position to a fitting release position against the biasing force to release the locked state of the elevating operation lever will be described. The same components and members as those of the endoscope 12 of the first embodiment are denoted by the same reference numerals and the descriptions thereof will be omitted.

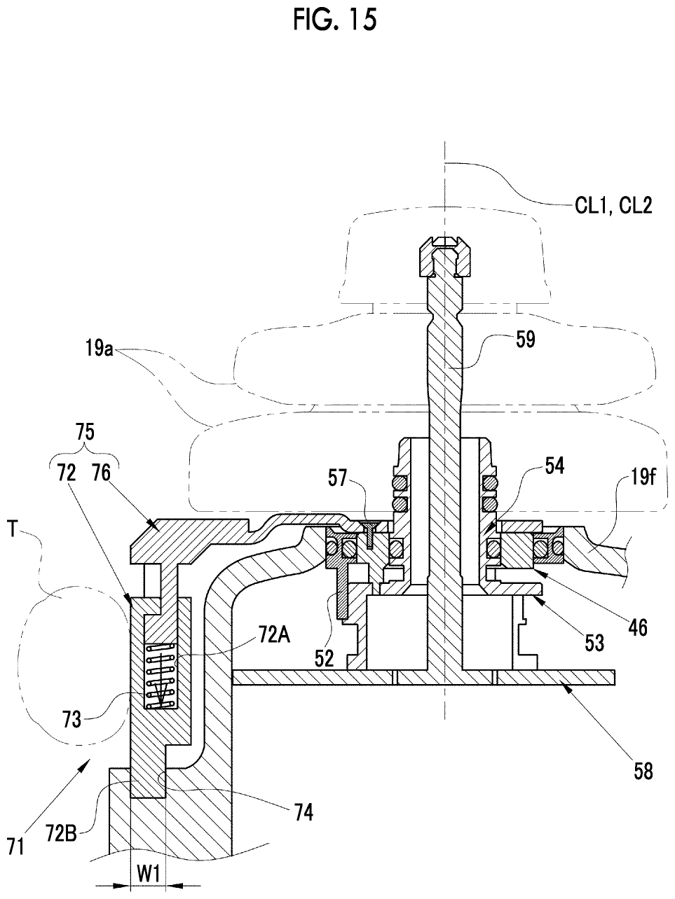

As shown in FIG. 15, a locking mechanism 71 includes a fitting member 72, a spring member 73, and a fitting groove 74. The fitting member 72 corresponds to the lock member and the pressing operation member in the claims. The fitting member 72 constitutes a part of an elevating operation lever 75. Similarly to the elevating operation lever 19b of the first embodiment, one end of the crank member 47 is rotatably connected to the elevating operation lever 75, and the elevating operation lever 75 constitutes the treatment tool elevating mechanism together with the rotary ring 46, the crank member 47, the guide barrel 48, the connection head 49, the slider 51, the fixed ring 52, the bearing member 53, and the like.

The elevating operation lever 75 includes the fitting member 72 and an elevating operation lever body 76. Similarly to the elevating operation lever 19b of the first embodiment, the elevating operation lever body 76 is connected to the rotary ring 46. Similarly to the finger hook member 56 of the first embodiment, the fitting member 72 is subjected to unevenness processing for forming unevenness on a front surface side on which the user hooks the finger.

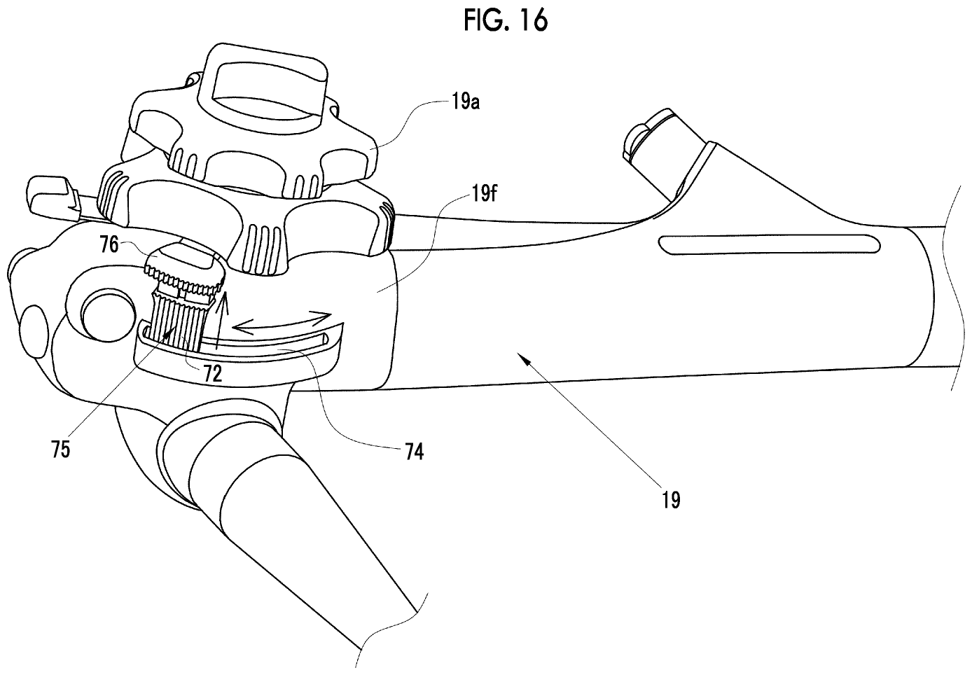

The fitting member 72 is attached to the elevating operation lever body 76 so as to be slidable along a direction parallel to the central axis CL1. The fitting member 72 is provided with a storage portion 72A and a fitting projection 72B. The fitting projection 72B is a projection extending in the direction parallel to the central axis CL1. The fitting groove 74 is an arc-shaped groove that is provided in the case 19f and is formed around the central axis CL1 (see FIG. 16). The fitting groove 74 is located on a distal end side of the fitting projection 72B, and is formed in accordance with a width W1 of the fitting projection 72B.

The spring member 73 is stored in the storage portion 72A in a state where one end of the spring member 73 is in contact with the fitting member 72 and the other end thereof is in contact with the elevating operation lever body 76. Accordingly, the spring member 73 applies a biasing force to the fitting member 72.

In a case where the elevating operation lever 75 is not operated, that is, in a case where the fitting member 72 is not pressed, the fitting member 72 is biased in the direction parallel to the central axis CL1 by the biasing force of the spring member 73, and the fitting projection 72B is fitted to the fitting groove 74. Accordingly, the elevating operation lever 75 is brought into the locked state. Hereinafter, a position where the fitting projection 72B is fitted to the fitting groove 74 is referred to as the fitting position or the lock position (a state shown in FIGS. 15 and 16).

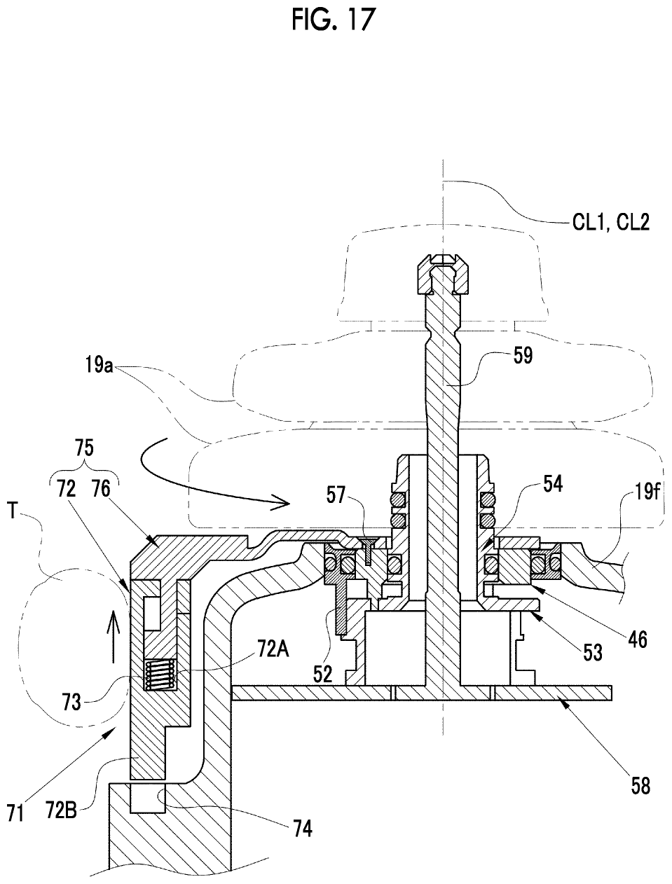

On the other hand, in a case where the elevating operation lever 75 is operated, the fitting member 72 is pressed to a proximal end side (upper side in FIG. 15) along the central axis CL1 as the lock release operation. The fitting member 72 is pressed and moved against the biasing force of the spring member 73. Therefore, the fitting projection 72B is separated from the fitting groove 74. Since the fitting projection 72B is released from the fitting groove 74, the locked state of the elevating operation lever 75 is released. Hereinafter, a position where the fitting projection 72B is released from the fitting groove 74 is referred to as the fitting release position or the lock release position (a state shown in FIG. 17).

The doctor can rotationally operate the elevating operation lever 75 without separating the thumb T after performing the lock release operation by pressing the fitting member 72 with the thumb T as described above, in a case of changing the lead-out direction of the treatment tool 13. That is, similarly to the first embodiment, the elevating operation lever 75 can be operated with one hand. Then, various treatments can be performed in a state where the lead-out direction of the treatment tool 13 is held in the desired direction.

### Third Embodiment

In the second embodiment, the endoscope comprises the locking mechanism 71 in which the fitting member 72 is fitted to the arc-shaped fitting groove 74 to bring the elevating operation lever 75 into the locked state, but the present invention is not limited thereto. In the third embodiment described below, an endoscope that comprises a locking mechanism in which a plurality of grooves are provided in the operation part and which is brought into the locked state in a case where a fitting member is fitted to any one of the grooves will be described. The same components and members as those of the endoscope 12 of the first embodiment are denoted by the same reference numerals and the descriptions thereof will be omitted.

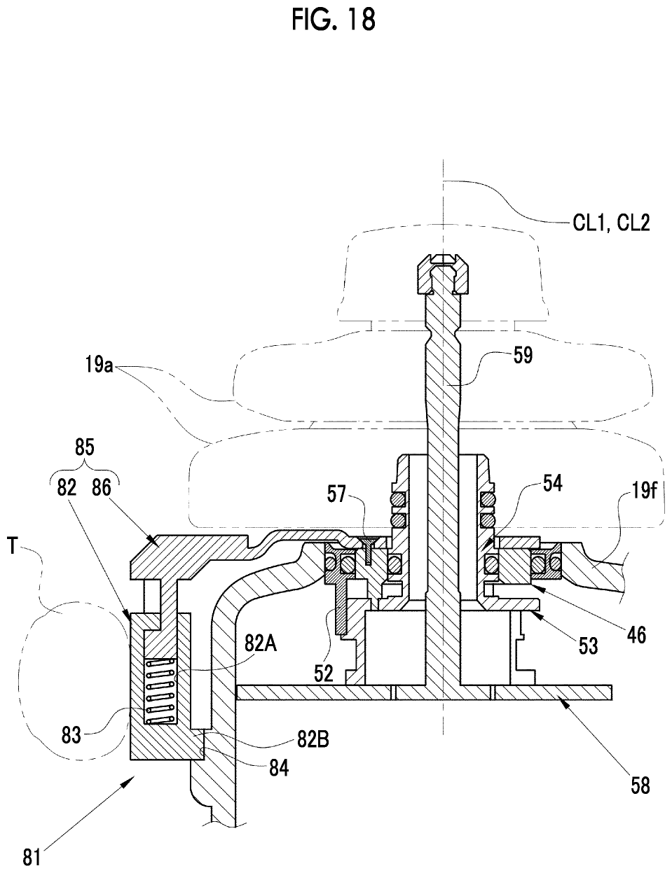

As shown in FIG. 18, a locking mechanism 81 includes a fitting member 82, a spring member 83, and a plurality of fitting grooves 84. The fitting member 82 corresponds to the lock member and the pressing operation member in the claims. The fitting member 82 constitutes a part of an elevating operation lever 85. Similarly to the elevating operation levers 19b and 75 of the first and second embodiments, one end of the crank member 47 is rotatably connected to the elevating operation lever 85, and the elevating operation lever 85 constitutes the treatment tool elevating mechanism together with the rotary ring 46, the crank member 47, the guide barrel 48, the connection head 49, the slider 51, the fixed ring 52, the bearing member 53, and the like.

The elevating operation lever 85 includes the fitting member 82 and an elevating operation lever body 86. Similarly to the elevating operation lever 19b and the elevating operation lever body 76 of the first and second embodiments, the elevating operation lever body 86 is connected to the rotary ring 46. Similarly to the finger hook member 56 of the first embodiment, the fitting member 82 is subjected to unevenness processing for forming unevenness on a front surface side on which the user hooks the finger.

The fitting member 82 is attached to the elevating operation lever body 86 so as to be slidable along a direction parallel to the central axis CL1. The fitting member 82 is provided with a storage portion 82A and a fitting projection 82B.

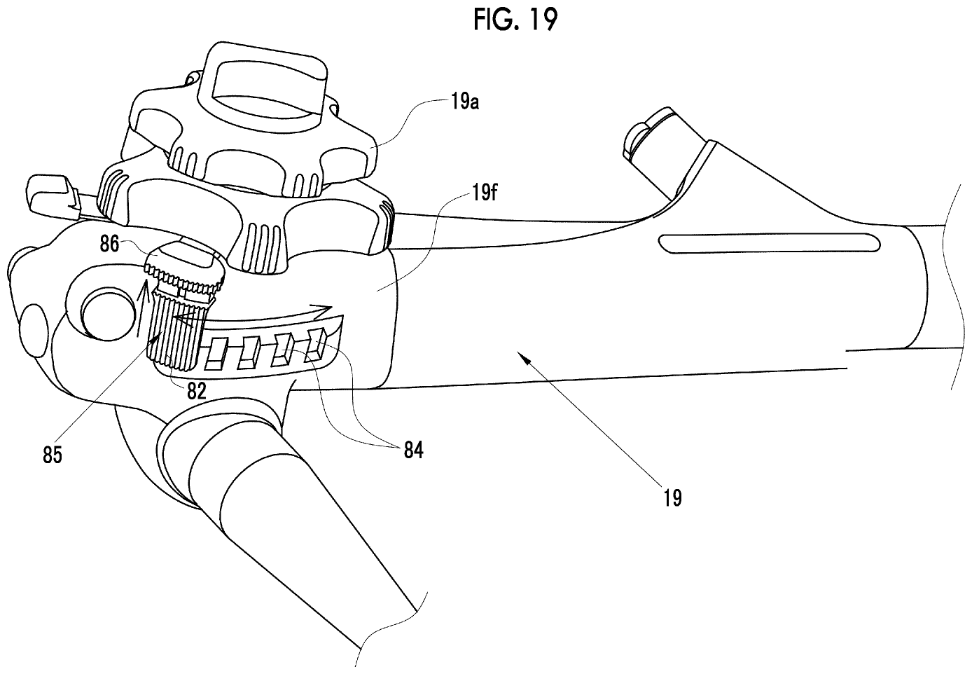

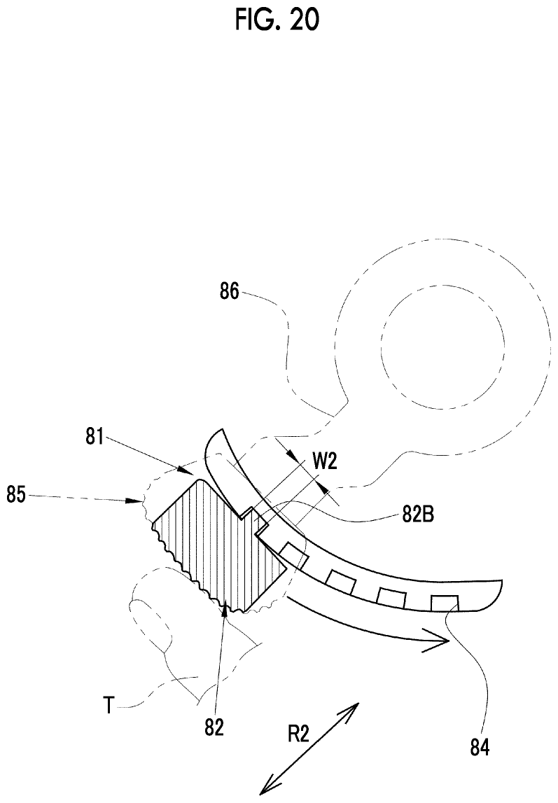

As shown in FIG. 20, the fitting projection 82B is a projection that protrudes in a radial direction R2 orthogonal to the central axis CL1. The fitting grooves 84 are a plurality of grooves provided in the case 19f and arranged around the central axis CL1 (see FIGS. 19 and 20). To be specific, the plurality of fitting grooves 84 are arranged at equal intervals along an arc located around the central axis CL1. The fitting grooves 84 is located on a distal end side of the fitting projection 82B, and are formed in accordance with a width W2 (see FIG. 20) of the fitting projection 82B.

The spring member 83 is stored in the storage portion 82A in a state where one end of the spring member 83 is in contact with the fitting member 82 and the other end thereof is in contact with the elevating operation lever body 86. Accordingly, the spring member 83 applies a biasing force to the fitting member 82.

In a case where the elevating operation lever 85 is not operated, that is, in a case where the fitting member 82 is not pressed, the fitting member 82 is biased in the direction parallel to the central axis CL1 by the biasing force of the spring member 83, and the fitting projection 82B is fitted to any one of the fitting grooves 84. Accordingly, the elevating operation lever 85 is brought into the locked state. Hereinafter, a position where the fitting projection 82B is fitted to the fitting groove 84 is referred to as the fitting position or the lock position (a state shown in FIGS. 18 and 19).

On the other hand, similarly to the elevating operation lever 75 of the second embodiment, in a case where the elevating operation lever 85 is operated, the fitting member 82 is pressed to a proximal end side (upper side in FIG. 18) along the central axis CL1 as the lock release operation. The fitting member 82 is pressed and moved against the biasing force of the spring member 83. Therefore, the fitting projection 82B is separated from the fitting groove 84. Since the fitting projection 82B is released from the fitting groove 84, the locked state of the elevating operation lever 85 is released. Hereinafter, a position where the fitting projection 82B is released from the fitting groove 84 is referred to as the fitting release position or the lock release position.

The doctor can rotationally operate the elevating operation lever 85 without separating the thumb T after performing the lock release operation by pressing the fitting member 82 with the thumb T as described above, in a case of changing the lead-out direction of the treatment tool 13. That is, similarly to the first embodiment, the elevating operation lever 85 can be operated with one hand. Then, various treatments can be performed in a state where the lead-out direction of the treatment tool 13 is held in the desired direction.

### Fourth Embodiment

In the second and third embodiments, the endoscope comprises the locking mechanisms 71 and 81 that are brought into the locked state in a case where the fitting member is fitted to the arc-shaped fitting groove 74 or the plurality of fitting grooves 84 arranged in the arc shape, but the present invention is not limited thereto. In the fourth embodiment described below, an endoscope that comprises a locking mechanism that is brought into the locked state in a case where a pressing operation member is pressed against an arc-shaped friction member will be described. The same components and members as those of the endoscope 12 of the first embodiment are denoted by the same reference numerals and the descriptions thereof will be omitted.

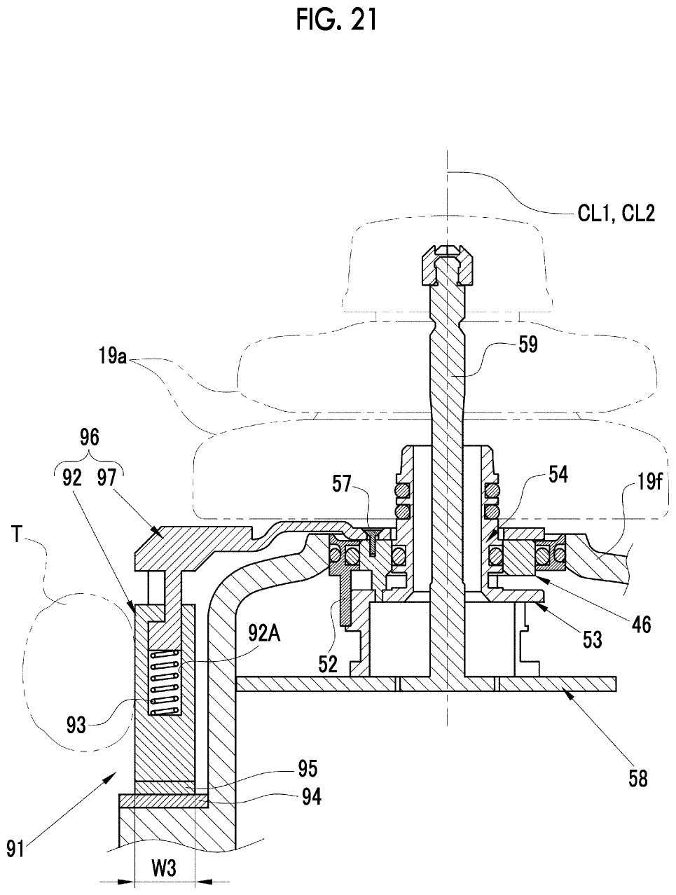

As shown in FIG. 21, the locking mechanism 91 includes a pressing operation member 92, a spring member 93, a first friction member 94, and a second friction member 95. The pressing operation member 92 corresponds to the lock member in the claims. The pressing operation member 92 constitutes a part of an elevating operation lever 96. Similarly to the elevating operation levers 19b, 75, and 85 of the first to third embodiments, one end of the crank member 47 is rotatably connected to the elevating operation lever 96, and the elevating operation lever 96 constitutes the treatment tool elevating mechanism together with the rotary ring 46, the crank member 47, the guide barrel 48, the connection head 49, the slider 51, the fixed ring 52, the bearing member 53, and the like.

The elevating operation lever 96 includes the pressing operation member 92 and an elevating operation lever body 97. Similarly to the elevating operation lever 19b and the elevating operation lever bodies 76 and 86 of the first to third embodiments, the elevating operation lever body 97 is connected to the rotary ring 46. Similarly to the finger hook member 56 of the first embodiment, the pressing operation member 92 is subjected to unevenness processing for forming unevenness on a front surface side on which the user hooks the finger. The pressing operation member 92 is attached to the elevating operation lever body 97 so as to be slidable along a direction parallel to the central axis CL1. The storage portion 92A is provided with the pressing operation member 92.

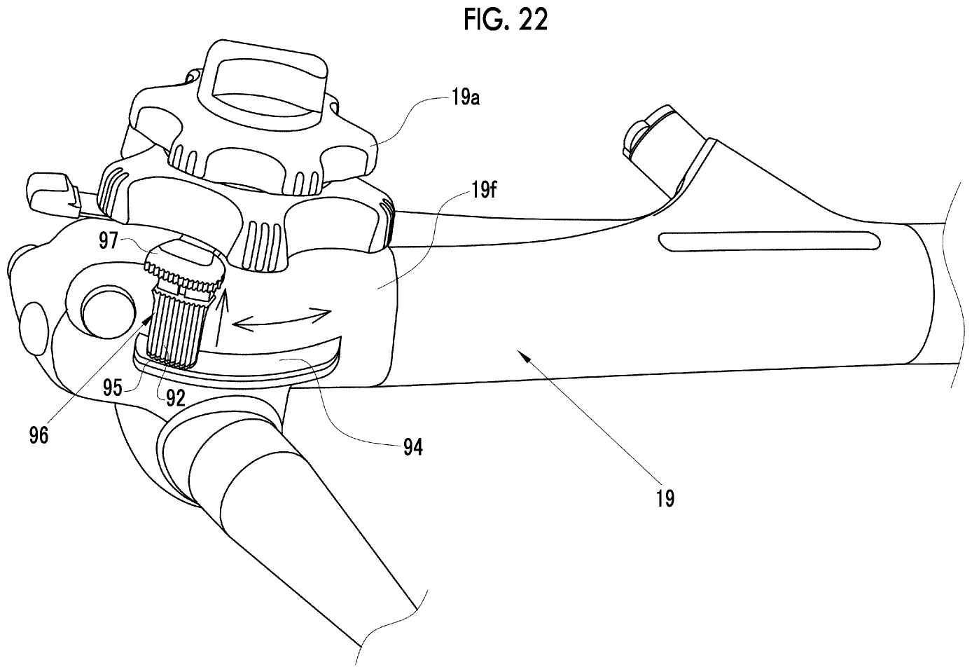

As shown in FIG. 22, the first friction member 94 is an arc-shaped friction member that is provided in the case 19f and is provided around the central axis CL1. The first friction member 94 is located on a distal end side of the pressing operation member 92, and is formed wider than a width W3 (see FIG. 21) of the pressing operation member 92.

The spring member 93 is stored in the storage portion 92A in a state where one end of the spring member 93 is in contact with the pressing operation member 92 and the other end thereof is in contact with the elevating operation lever body 97. Accordingly, the spring member 93 applies a biasing force to the pressing operation member 92. The second friction member 95 is provided at a distal end of the pressing operation member 92. The second friction member 95 is disposed at a position facing the first friction member 94. The pressing operation member 92 is moved toward the first friction member 94 by the biasing force of the spring member 93. Accordingly, the second friction member 95 brought into contact with the first friction member 94. The first and second friction members 94 and 95 are made of, for example, rubber or a soft resin.

In a case where the elevating operation lever 96 is not operated, that is, in a case where the pressing operation member 92 is not pressed, the pressing operation member 92 is biased in the direction parallel to the central axis CL1 by the biasing force of the spring member 93, and the second friction member 95 is brought into contact with the first friction member 94. Accordingly, the elevating operation lever 96 is brought into the locked state. Hereinafter, a position where the second friction member 95 is brought into contact with the first friction member 94 is referred to as the lock position (a state shown in FIGS. 21 and 22).

On the other hand, similarly to the elevating operation levers 75 and 85 of the second and third embodiments, in a case where the elevating operation lever 96 is operated, the pressing operation member 92 is pressed to a proximal end side (upper side in FIG. 21) along the central axis CL1 as the lock release operation. The pressing operation member 92 is pressed and moved against the biasing force of the spring member 93. Therefore, the second friction member 95 is separated from the first friction member 94. The locked state of the elevating operation lever 96 is released. Hereinafter, the position where the second friction member 95 is separated from the first friction member 94 is referred to as the lock release position.

The doctor can rotationally operate the elevating operation lever 96 without separating the thumb T after performing the lock release operation by pressing the pressing operation member 92 with the thumb T as described above, in a case of changing the lead-out direction of the treatment tool 13. That is, similarly to the first embodiment, the elevating operation lever 96 can be operated with one hand. Then, various treatments can be performed in a state where the lead-out direction of the treatment tool 13 is held in the desired direction.

In each of the above embodiments, a case where the treatment tool 13 is directed in the desired direction by rotating the elevator 33 from the fallen position toward the elevated position is described, but the present invention is not limited thereto, and even in a case where the elevator 33 is rotated from the elevated position toward the fallen position, the locking mechanism operates and the elevating operation lever can be brought into the locked state as in each of the above embodiments. Then, various treatments can be performed while the lead-out direction of the treatment tool 13 is held in the desired direction. In each of the above embodiments, a case where the first direction is the counterclockwise direction and the second direction is the clockwise direction is described, but the first direction may be the clockwise direction and the second direction may be the counterclockwise direction.

### EXPLANATION OF REFERENCES

10: endoscope system
12: endoscope
13: treatment tool
14: light source device
15: processor device
16: display
17: user interface (UI)
18: insertion part
18a: soft portion
18b: bendable portion
18c: distal end portion
18d: treatment tool channel
18e: treatment tool lead-out port
19: operation part
19a: angle knob
19b: elevating operation lever
19c: treatment tool lead-in port
19d: air and water supply button
19e: suction button
19f: case
21: flexible sheath
22: distal end portion
23: operation part
31: distal end portion body
32: cap
32A: peripheral surface portion
32B: end surface portion
32C: opening window
33: elevator
34: elevating operation wire
35: signal cable
36: light guide
37: disk portion
38: partition wall portion
39: partition wall portion
40: rotational shaft member
41: elevator housing portion
42: air and water supply nozzle
43: image sensor
44: illumination optical system
45: treatment tool elevating mechanism
46: rotary ring
47: crank member
47A: connection pin
48: guide barrel
49: connection head
51: slider
52: fixed ring
53: bearing member
54: rotational shaft
55: plate member
56: finger hook member
56A: storage portion
56B: through-hole
57: screw
58: frame member
59: attachment shaft
61: locking mechanism
62: locked member
62A: columnar portion
62B: locked claw
62C: protrusion
63: spring member
64: lock release button
64A: retaining portion
64B: pressing surface
65: locking piece
65A: recess
65B: locking piece
66: friction member
71: locking mechanism
72: fitting member
72A: storage portion
72B: fitting projection
73: spring member
74: fitting groove
75: elevating operation lever
76: elevating operation lever body
81: locking mechanism
82: fitting member
82A: storage portion
82B: fitting projection
83: spring member
84: fitting groove
85: elevating operation lever
86: elevating operation lever body
91: locking mechanism
92: pressing operation member
92A: storage portion
93: spring member
94: first friction member
95: second friction member
95: elevating operation lever
96: elevating operation lever
97: elevating operation lever body
CL1: central axis
CL2: central axis
CL3: central axis
R1: radial direction
R2: radial direction
T: thumb
W1: width
W2: width
W3: width

What is claimed is:

1. An endoscope comprising:
an insertion part that is to be inserted into a subject;
an operation part that is provided at a proximal end of the insertion part;
a distal end portion body that is located at a distal end of the insertion part, and communicates with a treatment tool lead-out port;
an elevator that is provided at the distal end portion body and causes a treatment tool led out of the treatment tool lead-out port to elevate, the elevator being provided to be movable between an elevated position and a fallen position;
an elevating operation lever that causes the elevator to move to the elevated position in accordance with rotation in a first direction and causes the elevator to move to the fallen position in accordance with rotation in a second direction; and
a locking mechanism that brings the elevating operation lever into a locked state,
wherein the locking mechanism includes:
a lock member that is provided in the elevating operation lever; and
a biasing member that applies a biasing force to the lock member, and
in the locking mechanism, in a case where the elevating operation lever is not operated, the lock member is biased to a lock position by the biasing force and the elevating operation lever is in the locked state, and in a case where the elevating operation lever is operated, the locked state of the elevating operation lever is released by a lock release operation against the biasing force.

2. The endoscope according to claim 1,
wherein the locking mechanism includes:
a locking piece that is provided in the operation part;
a locked member that serves as the lock member, the locked member being slidably attached to the elevating operation lever and being biased by the biasing force to a locked position where the locked member is locked to the locking piece; and
a pressing operation member that is provided integrally with the locked member, and
in a case where the pressing operation member is not pressed, the locked member is locked to the locking piece, and in a case where the elevating operation lever is operated, the pressing operation member is pressed against the biasing force to be moved integrally with the locked member and the locked member is moved from the locked position to a locked release position.

3. The endoscope according to claim 1,
wherein the locking mechanism includes:
a fitting groove that is provided in the operation part; and
a fitting member that constitutes a part of the elevating operation lever and is biased by the biasing force of the biasing member to a fitting position where the fitting member is fitted to the fitting groove, the fitting member being slidably attached to a body of the elevating operation lever, and
in a case where the fitting member is not pressed, the fitting member is fitted to the fitting groove, and in a case where the elevating operation lever is operated, the fitting member is pressed and moved against the biasing force and the fitting member is moved from the fitting position to a fitting release position.

4. The endoscope according to claim 3,
wherein the fitting member includes a fitting projection that extends in a direction parallel to a central axis of the elevating operation lever, and
the fitting groove is an arc-shaped groove that is fitted to the fitting projection and is formed around the central axis.

5. The endoscope according to claim 3,
wherein the fitting member includes a fitting projection that protrudes in a radial direction orthogonal to a central axis of the elevating operation lever, and
the fitting groove is a plurality of grooves that are fitted to the fitting projection and are arranged around the central axis.

6. The endoscope according to claim 1,
wherein at least some components are disposable.
